SEQUENCE LISTING (25 sequences)

SEQ ID NO: 1 - DNA, 408 bp, Neisseria gonorrhoeae
atgaatataatagaaataataagtaggaatcgttttctaaaacaaatatatcctagtggcataatggatatttcactagtctctttttcaactgacttgtctaattgtattttaactatccgaacaagtacaaagccttctgtagaaatcgaaaaatgggggctgtggctaaaagattatgatacagttgaaattgaattaagaaatagctttattaaaggaatgaaatgtcaaaattggtcgcataacaatagaaatatatgccaagtagaaataaagaaccaagaagatggtctaaaaataataagattttacgacaataattcaaattggttattggaactagaagtttatggattagttttccaagggtgtaagacttatatgaaagagggttatgaatcttaa

SEQ ID NO: 2 - DNA, 24 bp, Unknown
  Oligo
aattggtcgcataacaatagaaat

SEQ ID NO: 3 - DNA, 17 bp, Unknown
  Oligo
atttctattgttatgcg

SEQ ID NO: 4 - DNA, 34 bp, Unknown
  oligo
cttggcatatatttctattgttatgcgaccaatt

SEQ ID NO: 5 - DNA, 34 bp, Unknown
  oligo
cttggcatatatttctattgttatgcgaccactt

SEQ ID NO: 6 - DNA, 34 bp, Unknown
  oligo
cttggcatatatttctattgttatgcgtccactt

SEQ ID NO: 7 - DNA, 235 bp, Neisseria gonorrhoeae
ctgtagaaatcgaaaaatgggggctgtggctaaaagattatgatacagttgaaattgaattaagaaatagctttattaaaggaatgaaatgtcaaaattggtcgcataacaatagaaatatatgccaagtagaaataaagaaccaagaagatggtctaaaaataataagattttacgacaataattcaaattggttattggaactagaagtttatggattagttttccaagggtg

SEQ ID NO: 8 - DNA, 35 bp, Unknown
  oligo
gtatcataatcttttagccacagcccccatttttc

SEQ ID NO: 9 - DNA, 19 bp, Unknown
  oligo
gaaaaatgggggctgtggc

SEQ ID NO: 10 - DNA, 45 bp, Unknown
  oligo
tgtagaaatcgaaaaatgggggctgtggctaaaagattatgatac

SEQ ID NO: 11 - DNA, 35 bp, Unknown
  oligo
ctgtagaaatcgaaaaatgggggctgtggctaaaa

SEQ ID NO: 12 - DNA, 19 bp, Unknown
  oligo
ttttagccacagcccccat

SEQ ID NO: 13 - DNA, 35 bp, Unknown
  oligo
ctgtagaaatcgaaaaatcggggctgtggctaaaa

SEQ ID NO: 14 - DNA, 19 bp, Unknown
  oligo
ttttagccacagccccgat

SEQ ID NO: 15 - DNA, 35 bp, Unknown
  oligo
ttttagccacagcccccatttttcgatttctacag

SEQ ID NO: 16 - DNA, 19 bp, Unknown
  oligo
tacccccgacaccgatttt

SEQ ID NO: 17 - DNA, 19 bp, Unknown
  oligo
tagccccgacaccgatttt

SEQ ID NO: 18 - DNA, 22 bp, Unknown
  oligo
ctgtagaaatcgaaaaatgggg

SEQ ID NO: 19 - DNA, 25 bp, Unknown
  oligo
tcttggttctttatttctacttggc

SEQ ID NO: 20 - DNA, 34 bp, Unknown
  oligo
aattggtcgcataacaatagaaatatatgccaag

SEQ ID NO: 21 - DNA, 17 bp, Unknown
  oligo
cgcataacaatagaaat

SEQ ID NO: 22 - DNA, 10 bp, Unknown
  oligo
atatgccaag

SEQ ID NO: 23 - DNA, 16 bp, Unknown
  oligo
taaaagattatgatac

SEQ ID NO: 24 - DNA, 19 bp, Unknown
  oligo
gaaaaatgggggctgtggc

SEQ ID NO: 25 - DNA, 10 bp, Unknown
  oligo
tgtagaaatc

(19) United States
(12) Patent Application Publication
(10) Pub. No.: US 2017/0298424 A1
(43) Pub. Date: Oct. 19, 2017

(54) NUCLEIC ACID ANALYSIS

(71) Applicant: RevoluGen Limited

(72) Inventors: Stephen John MINTER, Hadfield, Derbyshire (GB); Georgios PATSOS, Hadfield, Derbyshire (GB)

(21) Appl. No.: 15/508,671

(22) PCT Filed: Sep. 4, 2015
(86) PCT No.: PCT/GB2015/052561

§ 371 Date: Jun. 5, 2017

(30) Foreign Application Priority Data
Sep. 4, 2014 (GB) 1415674.9

(51) Int. Cl. C12Q 1/68

(52) U.S. Cl. CPC C12Q 1/6823; C12Q 1/6897; C12Q 1/6818; C12Q 1/6827

(57) ABSTRACT

A method of analysing for a single stranded nucleic acid present, or potentially present, in a sample comprises a step in which the target nucleic acid (if present in the sample) displaces—and hybridises to—a reporter strand originally present in an interrogating duplex structure comprised of the reporter strand and a displaceable shorter strand. The reporter strand is tagged at or adjacent one end thereof with a reporter moiety capable of providing a detectable signal. The reporter/target duplex structure is such that the reporter strand may be selectively enzymatically digested (e.g. by means of λ-exonuclease) from its end opposite the reporter moiety to release that moiety for direct or indirect detection and regenerate single stranded target, which may then cycle through a plurality of the displacement and digestion steps to result in amplification of signal.

This application is the U.S. national phase of International Application No. PCT/GB2015/052561 filed 4 Sep. 2015, which designated the U.S. and claims priority to GB Patent Application No. 1415674.9 filed 4 Sep. 2014, the entire contents of each of which are hereby incorporated by reference.

The present invention relates to a method of analysing for the presence or otherwise of a particular (“target”) nucleic acid sequence in a sample, e.g. a liquid sample of biological origin, (e.g. a body fluid such as blood, urine, CSF or sputum, or one prepared from tissue, e.g. by homogenisation). The invention relates more particularly, but not necessarily exclusively, to such a method as applied to the diagnosis of a medical condition such as represented by the presence in the sample of the target nucleic acid sequence. The target nucleic acid sequence may, for example, be one having one or more point mutations as compared to a “wild-type” sequence.

The analysis of samples of biological origin to detect the presence of a particular nucleic acid sequence is a well establish science and is used routinely for the purposes of analysing liquid samples (e.g. a body fluid such as blood, urine, CSF or sputum, or one prepared from tissue, e.g. by homogenisation) to identify a particular medical condition. The medical condition may for example be an infection caused by a bacteria or virus that has “invaded” a patient's body and which may be characterised by the presence of a particular nucleic acid sequence in the infecting organism. A further possibility is the detection of a genetic disorder as characterised by one or more point mutations in a “wild-type” sequence present in a healthy individual. A still further possibility is for the case where a patient is known to have a certain medical condition but for which the treatment to be prescribed may be influenced by whether or not there is one or more point mutations in a nucleic acid sequence associated with the medical condition. Thus, for example, it is known that the mutation status of the K-ras gene in colorectal cancer may affect the patient's response to treatment with Cetuximab (a monoclonal antibody prescribed for patients who have a colorectal tumour and who have not responded to chemotherapy (Karapetis, C. S., et al 2008)).

Many such analyses of samples (e.g. blood, urine, CSF, tissue and sputum) to determine the presence, or otherwise, of a target nucleic acid sequence in the sample are routinely carried out by hospitals, doctors' surgeries and other medical centres every day and generally require that the patients' sample be sent to a laboratory (which may be on the premises of the medical centre) for an analysis procedure. Such analyses are frequently carried out using a procedure using amplification of the target nucleic acid sequence (if present) to detectable levels. qPCR is one such analysis method. However a disadvantage of such methods (i.e. those involving nucleic acid amplification), if not performed correctly, is the possibility of a mismatch that once formed results in an amplified negative (abortive) signal. A further disadvantage of such methods is that they frequently require lengthy gel development steps or column separation steps to obtain the result, which may then require skilled interpretation. Consequently some considerable time may elapse between the time when the sample is taken and the result is available to the medical practitioner who can then prescribe any necessary treatment.

As indicated, there is also a disadvantage associated with qPCR in that it is prone to “false positives” (i.e. indicating the presence of a particular nucleic acid in a sample when it is not so present) and may not always be capable of determining the presence of one or more point mutations in a nucleic acid sequence.

There is a need for a method of nucleic acid analysis which is easy to perform, which gives rapid and accurate results, and which is capable of being reliably used for the determination of point mutations in a target nucleic acid sequence. The present invention is addressed to this need.

According to the present invention there is provided a method of analysing for a single strand target nucleic acid sequence in a target nucleic acid present, or potentially present, in a sample the method comprising the steps of:
(i) providing an interrogating duplex nucleic acid structure which comprises
   (a) a first reporter strand which is specifically hybridizable to the single strand target nucleic acid sequence in the target strand (if present in the sample) and which is tagged at or towards one end thereof with a reporter moiety capable of providing a detectable signal, said reporter strand being configured such that in a reporter/target duplex structure formed by hybridisation of the first reporter strand and the single strand target nucleic acid sequence the reporter strand may be selectively enzymatically digested from its end opposite the reporter moiety to release the hybridised single strand target nucleic acid sequence and the reporter moiety; and
   (b) a second, displaceable strand shorter than said first reporter strand and hybridised thereto to form the interrogating duplex nucleic acid structure in which the reporter strand provides an interrogating overhang,
(ii) providing an enzyme capable of effecting said selective enzymatic digestion of the reporter strand in a duplex structure comprised of the reporter strand and target nucleic acid sequence, and
(iii) effecting the analysis under conditions such that the single strand target nucleic acid sequence, if present, displaces the second strand from the interrogating duplex nucleic acid structure and hybridises to the reporter strand with subsequent enzymatic digestion of the reporter strand, and
(iv) directly or indirectly detecting for the presence of reporter moiety.

As described more fully below, the method of the invention provides the significant advantage of signal amplification to provide a detectable signal from a low level of target nucleic acid sequence present in the sample without the need for amplification of the target sequence per se. Therefore, “false positives” resulting from mismatches during a nucleic amplification procedure are avoided. Furthermore, the method of the invention may be conducted in formats which provide a readily readable, and accurate detectable signal, which is obtained in a short period of time (e.g. 15-30 minutes depending on quantity of target nucleic acid strand (containing the target sequence) present in the sample and/or required sensitivity from the assay), thereby considerably reducing the time taken to achieve a result for the analysis, as compared to the prior art techniques discussed above.

In the method of the invention, the target nucleic acid strand (containing the target sequence) will generally be a DNA sequence but may be an RNA sequence. If the target nucleic acid strand is present, in the sample, in a double stranded structure then the target nucleic acid strand may be rendered single-stranded by conventional denaturation techniques which should be effected before introduction into the sample of the duplex nucleic acid structure (to prevent denaturation of the latter). Depending on the nature of the target nucleic acid strand, the reporter strand and the second displaceable strand may be DNA or RNA.

In preferred embodiments of the invention, the target, reporter and displaceable sequences will all be DNA sequences. However, we do not preclude other possibilities. For example, the target sequence may be DNA and the reporter sequence may be RNA. Alternatively, the target sequence may be RNA and the reporter sequence may be DNA.

In one embodiment of the invention, the target nucleic acid may be an amplicon produced in a nucleic acid amplification reaction (e.g. PCR). In a particular embodiment of the invention, a PCR reaction may be effected with primers such that one of the strands of the PCR product is digested by the same enzyme as used in step (ii) of the method of the invention, to leave the other (non-digested) strand as the target nucleic acid.

The sample will generally be a liquid and/or of biological origin.

In the method of the invention, the target nucleic acid sequence should be one which is unique in the analyte sample. The unique target sequence may be present within, and therefore as part of, a longer nucleic acid strand.

The reporter strand should be capable of hybridising to the target nucleic acid and in doing so hybridise along its (i.e. the reporter's full length) to the nucleic acid target strand in which the target sequence is present. Generally, the reporter strand will be shorter than the target nucleic acid strand in which the target sequence is present. The duplex nucleic acid structure is such that the nucleic acid strand containing the target sequence (if present) is capable of displacing the second shorter strand with the result that the reporter strand hybridises to the target nucleic acid sequence to form a target/reporter duplex.

The reporter strand has a reporter moiety at or adjacent one end of the strand. There may be more than one reporter moiety attached to the reporter strand. In certain embodiments of the invention, the reporter moiety may be a luminescent moiety. In other embodiments of the invention, the reporter moiety may be an enzyme.

The method of the invention employs enzymatic degradation of the reporter strand when hybridised to the target nucleic acid sequence from the end of the reporter strand opposite that at or adjacent which the reporter moiety is provided. The conditions should be such that neither strand of the interrogating duplex nucleic acid structure is digested to any significant extent compared to digestion of the reporter strand in the duplex structure formed by hybridisation of the reporter and analyte sequences. An efficient way of prevent digestion of the interrogating duplex structure is to make the overhang of the reporter strand (in the interrogating duplex structure) sufficiently long so there is only a low probability of digestion of the interrogating duplex (see discussion infra). Additionally, inhibition of digestion of the interrogating duplex structure may be achieved by appropriate configuration of the reporter strand, the configuration of the reporter/target duplex strand and/or the digesting enzyme (an exonuclease) that is used. Thus, for example, the nucleic acid structure in which the target sequence is present may be longer than the reporter strand and the 5′-end of the latter and the 3′ end of the former may form a blunt end in the reporter/target duplex (the reporter moiety being provided at the 3′ end of the reporter). The exonuclease may be one that preferentially digests in the 5′ to 3′-direction from a blunt end. Since the only blunt end in the analysis mixture is that provided by the reporter/target duplex (the 5′-end of the reporter being at the blunt end), the only significant strand digestion that takes place is that of the reporter strand.

In general terms, and assuming the presence of the target nucleic acid sequence in the sample being analysed, the method of the invention embodies the significant feature (described in more detail below) of signal amplification resulting from the following sequence of steps: (described in more detail below).
(i) displacement of the second, displaceable strand, i.e. a strand containing the target sequence, in the interrogating duplex nucleic acid structure by the target strand to form a reporter/target duplex;
(ii) selective enzymatic digestion of the reporter strand in the repoter/target duplex from one end thereof to provide free target strand and a “contribution” to a detectable signal (explained more fully below); and
(iii) recycling of free target strand to step (i).

In step (ii) above, the “contribution” to a detectable signal is achieved by virtue of the reporter moiety being released from the target/reporter duplex to participate in signal generation. Each repeat of step (i) and (ii) releases further reporter moiety. The reporter moiety may be attached to a terminal base of the reporter strand so that digestion of the full length thereof effects release of the reporter moiety. However we do not preclude the possibility that the reporter moiety is attached to a base inward of the end such that once the reporter strand has been digested as far as the base to which the reporter moiety is attached the remainder of the reporter strand is no longer capable of hybridisation, so that the reporter moiety is released.

It will be appreciated, therefore, that given the presence of the target nucleic acid strand in the sample and also a sufficient amount of the interrogating duplex nucleic acid structure (providing reporter strand with its associated reporter moiety), the target strands are capable of repeatedly cycling through steps (i)-(iii). Each repeat of step (ii) provides an additional “contribution” to the detectable signal resulting from recycling of the target nucleic acid strands, with the generation of an amplified signal.

Significantly, the signal amplification obtained as a result of the mechanism described in the previous paragraph is achieved without the need to amplify the target strand or the target sequence, thereby avoiding “false positives” obtained by such nucleic acid amplification procedures.

A further significant advantage of embodiment of the method of the invention lies in its specificity for detecting target nucleic acid sequences having one or more point mutations. Such embodiments utilise an exonuclease (for the purpose of digesting the reporter strand) for which the probability is that the digestion will only proceed to a base in the reporter strand which is non-complementary to the “opposite” base in the target sequence. Consider first and second target nucleic acid sequences that are identical to each other save for one base difference, in which case the second target sequence may be considered to be a point mutation of the first sequence. Consider now an analysis in which the first target sequence is present and is completely complementary to the reporter strand, with the reporter moiety being provided at or adjacent the opposite end of the reporter strand from that at which digestion commences. In this case, there is signal amplification as explained above. Consider now a further analysis procedure which uses the same reporter strand as the first procedure but in which the second target nucleic acid sequence is present. The second target nucleic acid sequence will be complementary to the reporter strand save at one location where there will be a mismatch. Given that the second target nucleic acid sequence and the reporter strand hybridise, then enzymatic digestion of the latter will commence as described above and will continue along the reporter strand up to the location of the mismatch. At this location, the probability is that enzymatic digestion of the reporter strand will cease or be delayed. Therefore, and depending on the number of mismatches in the second target nucleic acid, complete digestion of the reporter strand will be prevented, or at least delayed, so that the second target nucleic acid sequence remains part of a duplex structure (i.e. with the undigested portion of the reporter strand) and is not recycled (or is only recycled to a relatively low degree) for participation in signal amplification as described above.

Therefore the signal if any obtained from this second analysis is considerably lower than the (amplified) signal obtained from the first analysis.

To determine whether a sample of biological origin contains a target nucleic acid sequence which is a wild-type sequence or one which has a point mutation therein, then two analysis methods can be run, namely:
(a) one using a reporter strand having a sequence fully complementary to the wild-type target sequence; and
(b) one using a reporter strand having a sequence fully complementary with the target sequence having the point mutation.

If the target nucleic acid sequence in the sample is the wild-type sequence then analysis method (a) will give an amplified signal with there being significantly less, or no, signal from analysis method (b). Conversely, if the target nucleic acid sequence in the biological sample has the point mutation then the reverse is true, i.e. the signal from analysis method (b) is higher than that from method (a). On this basis, the method of the present invention is able readily to detect point mutations that may not be identified by qPCR.

One feature of the method of the invention is the interrogating duplex nucleic acid structure which comprises the reporter strand and second, shorter strand hybridised thereto such that the reporter strand has an “interrogating” overhang to which the target nucleic acid strand containing the target sequence will hybridise. The overhang should be sufficiently long that the target nucleic acid will hybridise to the reporter strand (with displacement of the shorter, second strand) but not so long that there is secondary structure in the overhang which might interfere with its “interrogating” function. The length of the overhang can also have an effect on the sensitivity of the method. If the overhang is too short, then there is an increased probability (as compared to a longer overhang) that the exonuclease will be able to digest the reporter strand in the interrogating duplex structure and release the reporter moiety, which therefore provides a contribution to the detected signal independent on the amount of analyte nucleic acid present in the sample. This effect can become significant in analyses where the amount of analyte sequence is very, very low, so that the contribution to the detected signal made by the analyte is low compared to the contribution made by digestion of the reporter strand in the interrogating duplex structure.

Ideally, the overhang formed by the reporter strand in the duplex structure will generally be at least 7 bases in length. However, the overhang should ideally be not more than 20 bases in length, more preferably not greater than 16. Preferably, therefore, the overhang should be 7 to 16 bases in length.

Generally, the reporter strand will be 24 to 35 bases in length.

Further, the duplex structure formed by hybridisation of the reporter strand to the target nucleic acid (containing the target nucleic acid sequence) must be such that the reporter strand is capable of being selectively enzymatically digested by an enzyme (exonuclease) provided for this purpose so as to release the target strand for use in further rounds of displacement of the second, shorter strand of the interrogating duplex nucleic acid structure to provide for amplification as described above. The exonuclease is preferably one capable of effecting the digestion isothermally, ideally at a temperature of 15 to 40° C., e.g. 35 to 40° C., e.g. about 37° C.

For preference, the reporter/target duplex structure may be one having a blunt end configured for digestion of the reporter strand, by the enzyme, from that blunt end. Most preferably, the target nucleic acid strand (containing the target nucleic acid sequence) is longer than the reporter strand so that there is only one blunt end in the reporter/target duplex, with the target nucleic acid providing a (single-strand) “tail” for the duplex structure. This “tail” may be up to about 200 bases in length (e.g. 100-200 bases). If any longer than 200 bases then there may be a residual interaction between the tail and the enzyme molecule which prevents re-use of that enzyme in further rounds of strand digestion. In a preferred embodiment of the invention, the reporter strand has a 5′-phosphorylated end (at the blunt end of the duplex structure) and the enzyme is A-exonuclease which processively degrades one strand of double stranded DNA in the 5′-3′ direction in the following order of preference for the configuration of the ends of the double stranded structure, namely 5′-recessed>blunt>>5′-overhang with a 10× preference for phosphorylated rather than hydroxylated ends. Further, 3′ tails>100 bases in length are known to inhibit enzymatic activity of A-exonuclease (Mitsis and Kwagh, 1999). Therefore, when A-exonuclease is used, any “tail” in the duplex structure is no longer than 100 bases in length. In this embodiment of the invention, digestion of the reporter strand in the reporter/target duplex structure proceeds from the 5′-phosophyrlated end of the reporter strand to digest the latter and release the target nucleic acid strand for further rounds of displacement to provide amplification, as discussed more fully above.

With specific reference to the use of A-exonuclease in the method of the invention (and to expand on the possibility of digestion of the interrogating duplex structure which might provide a reduction in sensitivity of the analysis), there are two reactions in the analysis potentially proceeding in parallel (at least for the case where the interrogating duplex structure is not immobilised (see infra).

The first is that the A-exonuclease digests the reporter strand in the duplex structure formed by hybridisation of the reporter and analyte strands.

The second is that the enzyme may also digest the reporter strand in the interrogating duplex structure. This could limit sensitivity, because at relatively low amounts of analyte (zmol to amol) most of the enzymatic digestion might be of the interrogating duplex structure (which may be present in pmol quantities) and would not allow for very small amounts of analyte to be detected.

Another issue regarding this system is that although the digestion is isothermal and can be carried out at ambient temperature, the closer the temperature is to 37° C. the more active the enzyme will be and the more background digestion will be generated. Since the A-exonuclease can only digest from a 5′ to 3′ direction, it either digests the reporter strand starting on its 5′-phosphorylated end or the displaceable oligonucleotide starting on the 5′-end thereof at which the reporter quencher is provided. It is unlikely that the enzyme will digest the 5′-“quencher moiety containing end” of the displaceable oligonucleotide even though it creates a blunt end because the quencher moiety will generally be a relatively large molecule and would create steric hindrance. It is more likely that the A-exonuclease recognises the 5′-phosphorylated end of the reporter strand and digests that strand to release the reporter moiety that generates a background signal. It is however known that a single stranded 5′-phospolyrlated end can bind to the active site of the A-exonuclease. If this is the case then the A-exonuclease could be brought into proximity with the double stranded part of the interrogating duplex structure and this will start background digestion thereof leading to a background signal.

In theory, increasing the size of the overhang provided by the reporter strand should (i) place the 5′-phosphorylated end at a “safe distance” from the double stranded duplex of the interrogating duplex structure so even if the A-exonuclease binds to the overhang of the reporter strand it will not recognise a double stranded molecule to digest, or (ii) the overhang will be more flexible and therefore less likely to bind to the enzyme (this enzymes' preference for double stranded molecules could relate to target flexibility/stability), or (iii) a longer overhang would create a hairpin that could still allow displacement by the analyte strand to occur but would block enzyme binding and digestion.

In practice, use of an overhang of about 16 bases and carrying out the reaction under highly active conditions (37° C. or 40° C., 10 U) reduces background digestion from tens of thousands of RFU (Relative Fluorescence Units) to a few a hundred after an hour of incubation, and in this way the sensitivity of the system increases because enzymatic activity will predominantly focus on a duplex structure formed by hybridisation of the reporter and analyte strands rather than the interrogating duplex structure.

In the case where the enzyme is A-exonuclease, the target strand may be one produced in a PCR reaction in which one of the primers has a 5-phosphorylated end. In the double-stranded PCR product obtained, the strand having the 5-phosphorylated end may be digested by the A-exonuclease to leave the other (non-digested) strand as the target nucleic acid.

A further exonuclease that may be used in the method of the invention is exonuclease III.

Exonuclease III catalyzes the stepwise removal of mononucleotides from 3″-hydroxyl termini of duplex DNA (Roger and Weiss, 1980). The enzyme is not active on single-stranded DNA, and thus 3″-protruding termini are resistant to cleavage (New England Biolabs website). Therefore an interrogating duplex can be designed where the overhang of the reporter will be on the 3′ end and because the overhang is single stranded it will not be digested unless an analyte single stranded DNA molecule hybridizes to the overhang, and displaces the reporter creating a blunt 3′ end which will then be the target for exonuclease III digestion leading to the release of the analyte due to reporter digestion.

The nucleic acid strand containing the target sequence may be one excised using standard restriction enzyme techniques from a much longer length of a naturally occurring double stranded sequence such that the excised nucleic acid strand has an appropriate length and also contains the target nucleic acid sequence. The excision of a double strand sequence of appropriate length and its conversion into single strands for use in the method of the invention may be effected by use of restriction enzymes and denaturing techniques as well known in the art. Thus, for example, it is possible to prepare the analyte strand that can be used in the displacement amplification system by following these steps.

1. Endonuclease digestion of genomic DNA that will generate a dsDNA of the appropriate sequence (therefore sequence selection for this system depends on endonuclease site availability and endonuclease buffer compatibility when using two enzymes).
2. Denaturation of dsDNA endonuclease product and capture of analyte sequence on biotinylated oligonucleotide that is bound to streptavidin magnetic beads.
3. Collect beads on magnets and wash to remove irrelevant DNA sequences.
4. Denaturation of analyte sequence off the biotinylated oligonucleotide that is bound to the magnetic bead and use of eluate as analyte.

There is also an alternative way of using this particular displacement amplification system to produce a specific signal using genomic DNA as analyte.

An alternative procedure for obtaining single stranded DNA for conducting an analysis method in accordance with the invention and (in this case) obtaining an at least semi-quantitative result is as follows.

(a) Two aliquots of identical volume are obtained from a liquid sample containing (or potentially containing) a double-stranded DNA sequence to be analysed for. For convenience, the two aliquots are designated herein as the “first” and “second” aliquots.
(b) The first aliquot is subjected to heat denaturation in the presence of a specific amount of a first oligonucleotide that will hybridise to a sequence in the (longer length) single stranded DNA. The mixture is then renatured with hybridisation of the first oligonucleotide to the single stranded DNA of interest (if present) under conditions that facilitate hybridisation (e.g. presence of MgCl₂). The non-hybridised first oligonucleotide serves as a single stranded target nucleic acid sequence in step (d) below.
(c) Step (b) is repeated for the second aliquot but using an equimolar amount (compared to the first oligonucleotide) of a second oligonucleotide that is non-specific to the DNA in the sample. The non-hybridised second oligonucleotide serves as a single stranded target sequence in step (e) below. The second oligonucleotide may, for example, be a salmon DNA sequence.
(d) The mixture from step (c) is analysed according to the method of the invention using an interrogating duplex structure in which the reporter will hybridise to the single-stranded (i.e. non-hybridised) first oligonucleotide in the sample. The signal obtained is representative of the amount of the non-hybridised, first oligonucleotide.
(e) The mixture from step (c) is analysed in accordance with the method of the invention using an interrogating duplex nucleic acid structure in which the reporter will hybridise to the second oligonucleotide. The signal generated is representative of the amount of non-hybridised second oligonucleotide (e.g. salmon DNA) in the mixture.
(f) The difference in signals obtained in steps (d) and (e) is representative of the amount of DNA of interest in the original liquid sample.

The procedure just described assumes that the amplified signal generated by a given amount (e.g. 100 fmol) of each of the first and second oligonucleotides against their respective interrogating duplex structures will be identical or very similar. One way to ensure this will happen is by choose oligonucleotide sequences with the same (or very similar) thermodynamic properties. For example, it is possible to design sequences that will have the same or very similar GC content for the full length of the reporter sequence, the overhang sequence and the sequence of the reporter that will hybridise to the displaceable oligonucleotide.

The method of the invention may be effected using conventional aqueous buffers. However, if the enzyme is A-exonuclease, it is preferred that the buffer is other than conventional Phosphate Buffered Saline (PBS). In particular, it is preferred that (for A-exonuclease) the buffer is a phosphate-based aqueous buffer which contains relatively low concentrations of sodium and chloride ions (if present at all). Preferably the buffer has a concentration of less than 80 mM of each of sodium and chloride ions, more preferably less than 60 mM, and even more preferably less than 40 mM. The sodium ion concentration may be equal to or greater than the chloride ion concentration. For example, the buffer may have a concentration of 20-50 mM (e.g. 35-45 mM) sodium ions and 2-7 mM (e.g. 4-6 mM) chloride ions. Suitable buffers may, for example, be formulated from Na₂HPO₄ and KCl in appropriate amounts. The buffer may contain a surfactant, e.g. Tween.

The method of the invention may be effected either in solution phase or using a solid phase support in, for example, a flow based assay in which the second oligonucleotide of the interrogating duplex nucleic acid structure is immobilised in a flow pathway (e.g. a capillary flow pathway).

In the case of a solution phase reaction, the signal provided by the reporter moiety may be detected in the solution phase of the reaction. In this case, the signal (determinative of the presence in the sample of the target nucleic acid sequence) may be detected in the liquid phase. For this embodiment, it is important that no signal is generated unless the target nucleic acid is present in the sample and has been able to participate in the sequence of reactions summarized as (a)-(c) above otherwise a false result would be obtained. Thus, in such a solution phase assay, signal generation by the reporter moiety must be inhibited until this sequence of reactions has occurred. This may most conveniently be effected by use of a luminescent (fluorescent or phosphorescent) reporter moiety on the reporter strand and a quencher on the second, shorter strand in the interrogating duplex nucleic acid structure which quenches the luminescence until the second strand is displaced from the reporter strand by target nucleic acid.

In the case of a flow (e.g. capillary flow) based assay, this may be effected using an assay device having a “reaction region” and a “detection region”. The “reaction region” comprises the interrogating duplex nucleic acid structure with its shorter (second) strand immobilised on and/or around the capillary pathway. Liquid flows from the “reaction region” to the “detection region” where measurement of signal generated by the reporter moiety may be measured. Since the detection region is separate from the reaction region there is no need for masking of the reporter moiety. Thus, the reporter moiety attached to the reporter strand may be a non-quenched luminescent reporter moiety (which is only released to pass to the detection region if the reaction sequence (a)-(c) has occurred). Alternatively, the reporter moiety may be one which is not detected per se but rather is “indirectly” detected by the product of a reaction in which the released reporter moiety participates with an appropriate reagent/substrate, e.g. provided at the detection region. Thus, for example, the reporter moiety may be an enzyme which generates a colour with an appropriate substrate. The enzyme may, for example, be alkaline phosphatase or Horse Radish Peroxidase for which appropriate substrates are shown in Table 1 below.

TABLE 1

| Enzyme | Substrate for signal generation |
|---|---|
| Alkaline Phosphatase | 1 2 Dioxoetane (chemiluminescent) |
| | D luciferin-O-Phosphate (Bioluminescent) |
| | BCIP/NBT - Blue colour |
| Horse Radish Peroxidise | Luminol |

The invention will now be further described, by way of example only, with reference to the accompanying drawings and non-limiting Example. In the drawings:

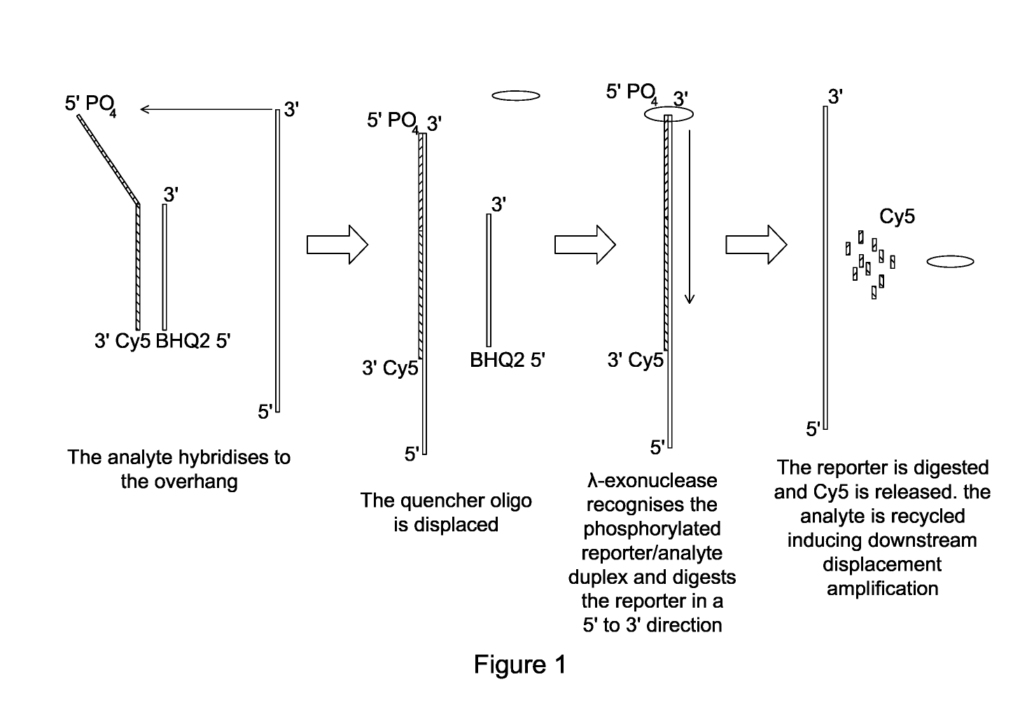

FIG. 1 schematically illustrates one embodiment of the method of the invention effected as a solution phase analysis;

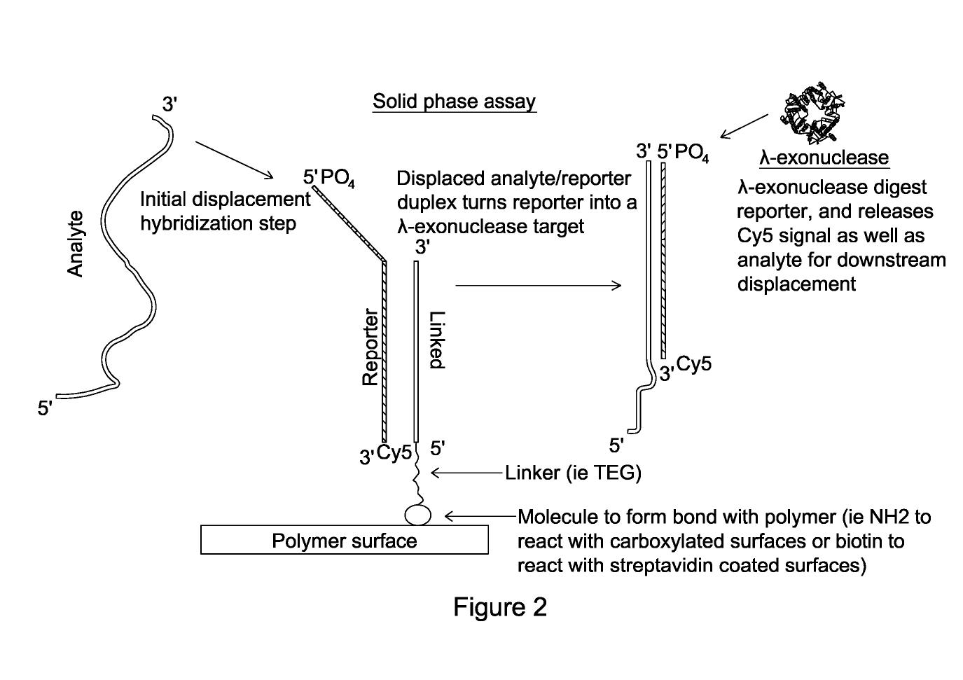

FIG. 2 schematically illustrates a further embodiment of the method of the invention effected using a solid phase support;

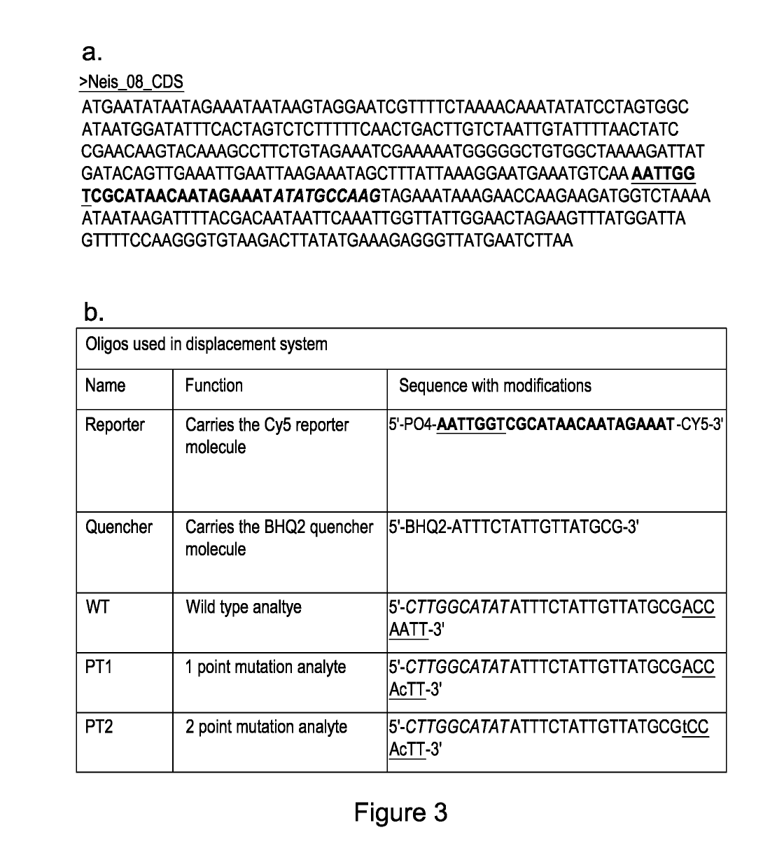

FIG. 3 shows details, in the order indicated, of nucleotide sequences represented as SEQ ID NOS: 1-6 employed in Example 1;

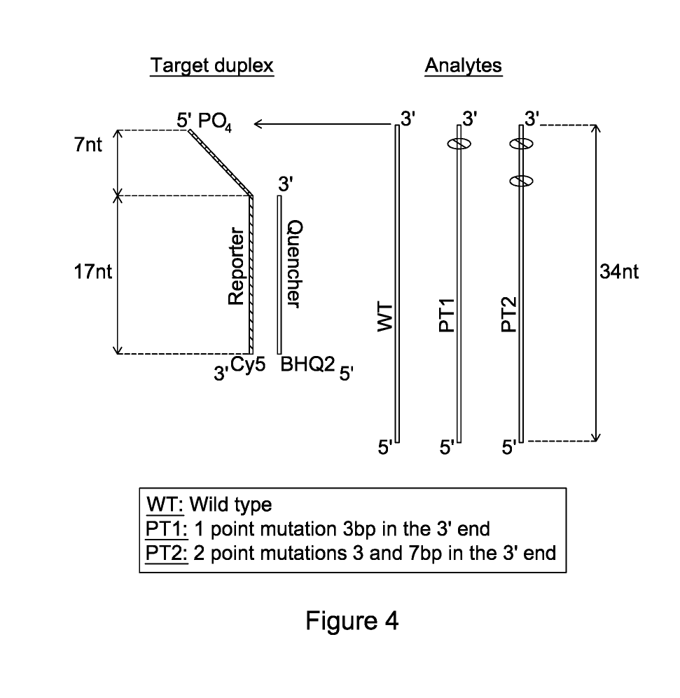

FIG. 4 schematically illustrates the method of Example 1; and

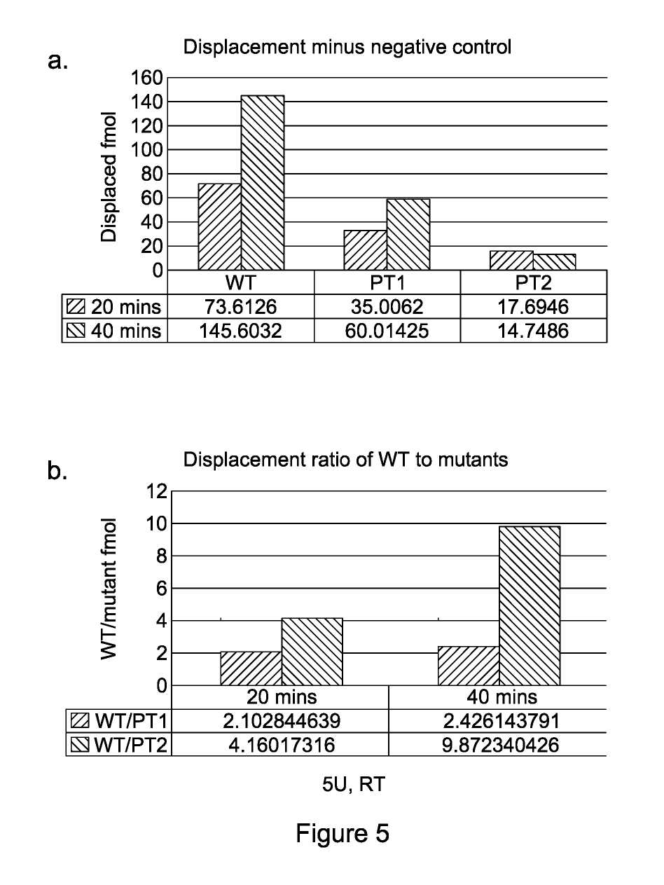

FIG. 5 illustrates the results of the Example 1;

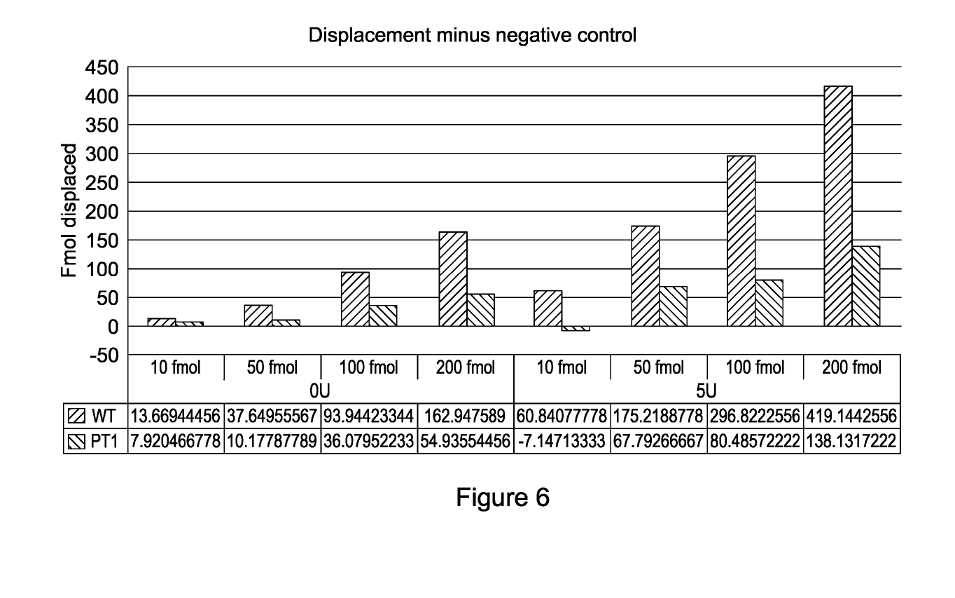

FIG. 6 illustrates the results Example 2;

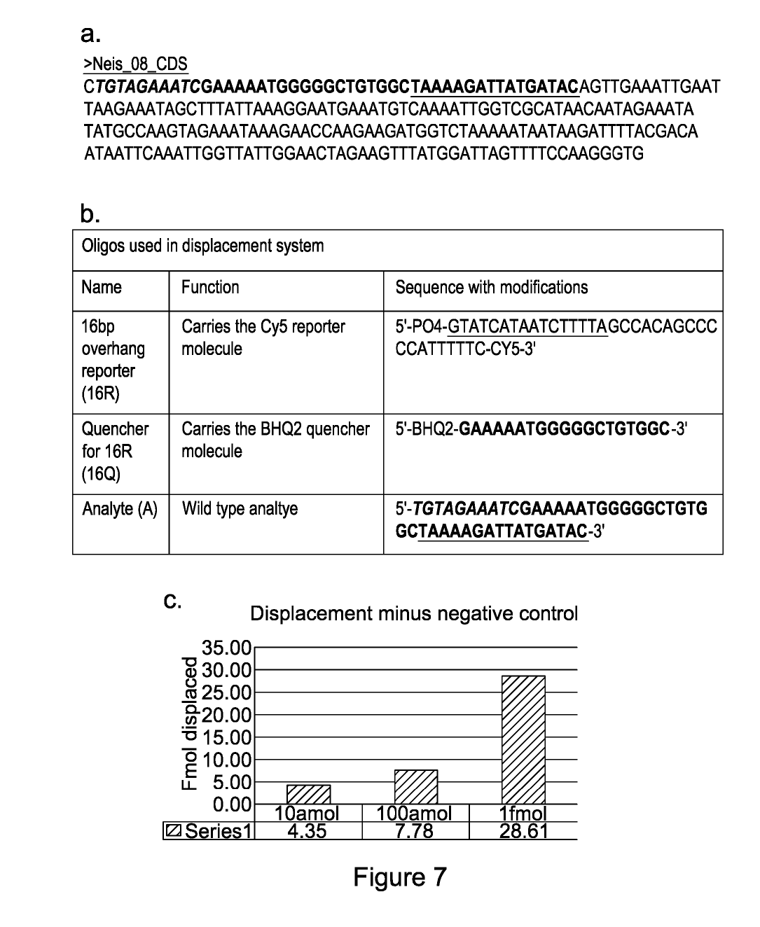

FIGS. 7(a) and (b) show details, in the order indicated, of sequences represented as SEQ ID NOS: 7-10 employed in Example 3; and

FIG. 7(c) illustrates the results of Example 3.

Reference is firstly made to FIG. 1 which shows a solution phase embodiment of the analysis method of the invention. More specifically, FIG. 1 (a) illustrates an interrogating duplex nucleic acid structure which is comprised of two nucleic acid (DNA) strands hybridised together. More particularly, the interrogating duplex nucleic acid structure comprises:
(a) a first reporter strand which is phosphorylated at its 5′-end and provided with a fluorescent label (Cy5) at its 3′-end; and
(b) a second shorter strand having a sequence complementary to the 3′ end of the reporter strand and being provided at its 5′ end with a BHQ2 (“Black Hole Quencher 2”) moiety at its 5′ end.

As it will be appreciated from FIG. 1(a) the 5′-region of the reporter strand provides an overhang in the interrogating duplex nucleic acid structure, the purpose of which will be described more fully below.

The BHQ2 moiety serves to quench the fluorescence of the Cy5.

Further illustrated in FIG. 1(a) is a single stranded, nucleic acid (DNA) target sequence, shown as having a length greater than the reporter strand. For the purposes of the present description, the reporter strand and the target strand are considered to be fully complementary to each other reading from their 5′ and 3′-ends respectively.

Further provided in the liquid phase is the enzyme A-exonuclease. The A-exonuclease is not shown in FIG. 1(a) but is illustrated as an ellipse in FIGS. 1(b)-(d).

Conveniently, the method may be affected by “pre-preparing” the interrogating duplex nucleic acid structure in solution. This solution itself may then be used for the analysis procedure by incorporating the sample to be analysed and the A-exonuclease into the solution. Alternatively, the solution comprising the interrogating duplex nucleic acid structure may be lyophilised for incorporation in a well of an analysis device (e.g. a microtitre plate) in which the analysis is carried out by formation of a liquid mixture containing (or potentially containing) the target sequence and the A-exonuclease.

The reaction proceeds under hybridising conditions so that the target sequence displaces the quencher oligonucleotide from the reporter and hybridises thereto, as schematically illustrated in FIG. 1(b). In view of their complementary sequences, the reporter and the target hybridised to provide a duplex structure with a blunt end formed by the 5′ end of the reporter and the 3′ end of the target (see FIG. 1(b).

Given that the reporter has a 5′ phosphorylated end at a blunt end of the duplex structure, the λ-exonuclease now proceeds selectively to digest the reporter strand from its 5′ end, as depicted in FIG. 1(c) by the exonuclease (depicted as an “ellipse”) passing over and along the duplex structure. With full digestion of the reporter strand, the original target strand, the λ-exonuclease molecule used for digestion, and the Cy5 originally bound to the reporter moiety are now all free in the liquid phase.

The net effect is that the target strand (and the λ-exonuclease) are free to undergo a further sequence of reactions as depicted by FIGS. 1(a)-(d). The overall effect, therefore, is one of signal amplification—namely that each target displaces more than one Cy5 allowing amplification of the fluorescence signal, as compared to the case where the exonuclease is not provided in the analysis mixture (in which case each target strand only serves to displace one quencher strand and thereby only release one Cy5 from its associated BHQ2 quencher).

This signal amplification is a significant feature of the invention since it allows detection of very low levels of target molecules which (without the amplification provided by the method of the invention) could be insufficient to provide a detectable signal.

The solution phase embodiment has been described above with specific reference to the reporter moiety being Cy5 and the quencher being BHQ2. It will however be appreciated that other combinations of fluorescent reporter moiety and quencher may be used. Examples of combinations of fluorescent reporter moiety and quencher that may be used are shown in the following table:

Reporter/Quencher
Cy5/BHQ2
Cy5/BHQ1
TexRed/BHQ2
TexRed/QSY7
TexRed/BHQ1
TexRed/Dabcyl
Cy3/BHQ2
Cy3/BHQ1
FAM/BHQ2
FAM/QSY7
FAM/BHQ1
Quasar670/BHQ2
CallRed/BHQ2
Quasar570/BHQ2
TAMRA/BHQ2
TAMRA/Dabcyl
FAM/BHQ2

It will however be appreciated that the solution phase analysis as described above will generally require the use of a quencher moiety, in conjunction with the fluorescent reporter moiety. This is of course, to ensure, that in the absence of target nucleic acid, no fluorescence will be generated (since fluorescence emission is dependent on the quencher strand being displaced (by the target nucleic acid) from the fluorescent reporter moiety.

Detection of fluorescent emission from the Cy5 or other fluorescent reporter moiety that may be used may be detected in the solution phase by any conventional means with the intensity of the (amplified) fluorescence being a quantitative measure of the amount of target nucleic acid in the original sample.

Reference is now made to FIG. 2 which illustrates a further embodiment of method in accordance with the invention but, in this case, effected using a solid phase support. Preferably, the embodiment of FIG. 2 is effected in an assay device having a flow (e.g. capillary flow) pathway with an upstream sampling region, and a downstream detection region and an intermediate “reaction region” in which the interrogating duplex nucleic acid structure is immobilised, more particularly by virtue of its second strand being linked to (and therefore immobilised on) the solid phase. Similar such assay devices (together with techniques for immobilising nucleic acids on the capillary pathway thereof) are disclosed in WO 2012/049465.

In such an assay device, a liquid sample (potentially) containing the target nucleic acid sequence is introduced onto the sampling region and is then able to flow (e.g. by capillary action) to a region of the flow (e.g. capillary flow) pathway at which the interrogating duplex nucleic acid structure is immobilised, the flow then continuing to the downstream detection region, which for convenience is a well into which the liquid flows for the purpose of detection signal.

In principle, the embodiment of FIG. 2 functions in a manner entirely analogous to that described in FIG. 1 to the extent that the target nucleic acid sequence displaces the reporter sequence and hybridises thereto to form a “blunt ended” duplex structure for which the reporter strand is then digested (from its phosphorylated 5′-end) by the λ-exonuclease to release the target nucleic acid strand which is then able to partake in further displacement reactions (i.e. displacement of the reporter strand from the interrogating duplex nucleic acid structure). However, as applied to a flow (e.g. capillary flow) based procedure in which the interrogating duplex nucleic acid structure is immobilised, there are the following differences as compared to the embodiment of FIG. 1.

Firstly, as indicated above, the interrogating nucleic acid structure is immobilised on the (inner) wall of the flow (e.g. capillary flow) pathway. Although only one such immobilised interrogating duplex nucleic acid structure is illustrated in FIG. 2, there will be many such structures immobilised along the length, and around the interior, of the flow pathway.

Secondly, the second strand (i.e. the strand that is immobilised on the wall of the flow pathway) does not carry a quencher. Put another way, there is no quenching of the Cy5 or other fluorescent reporter moiety in the interrogating duplex nucleic acid structure.

Thirdly, it is possible for the reporter moiety to be an enzyme (e.g. Alkaline Phosphatase or Horse Radish Peroxidase) which develops a colour by reaction which a substrate provided at the detection region.

In an alternative embodiment of the method of the invention using a solid phase support, the latter could, for example, be the internal surface of a well of a microtitre plate on which the interrogating duplex nucleic acid structure is immobilised. For this embodiment, the reporter moiety should be a quenched luminescent moiety, as described for the embodiment of FIG. 1.

The invention will now be illustrated with reference to the following non-limiting Examples.

### EXAMPLE 1

### General Description

This Example demonstrates use of the method of the invention for detecting a 34 nucleotide target sequence from Neisseria Gonorrhoeae CDS 8 in a liquid sample, the method employing a fluorescent label for the purpose of detecting the presence of the nucleic acid. More particularly, the Example demonstrates amplification of the fluorescent signal as compared to a control method not embodying the invention. Additionally, the Example demonstrates the specificity potential of the method of the invention for distinguishing between a wild-type nucleic acid sequence and a similar sequence with at least one point mutation.

Reference is firstly made to FIGS. 3a and 3b. FIG. 3a shows a conserved sequence in Neisseria Gonorrhoeae CDS 8 incorporating a unique 34 nucleotide sequence highlighted in bold. This 34 nucleotide sequence is currently used in a qPCR method for the detection of Neisseria Gonorrhoeae CDS 8.

FIG. 3b shows nucleotide sequences (based on the unique sequence) which were used as “reporter”, “Quencher”, “Wild Type” (“WT”) analyte “1 Point Mutation” (“PT1”) analyte and “2 Point Mutation” (“PT2”) analyte sequences used for the purpose of this Example.

The WT analyte sequence comprised all 34 nucleotides of the unique sequence (i.e. the emphasised sequence—bold or underlined—in FIG. 3(a)). In PT1 and PT2, the mutation(s) is/are represented in lower case letters. Compared to WT, (a) PT1 had one point mutation (C instead of A) three nucleotides from its 3′-end, and (b) PT2 had the same mutation as PT1 and an additional mutation (T instead of A) seven nucleotides from its 3′-end.

Reading from its 5′-end, the reporter sequence comprised 24 nucleotides which provided a complementary sequence to the first 24 nucleotides reading from the 3′ end of the WT sequence. As shown in FIG. 3b, the reporter sequence was phosphorylated at its 5′-end and carried a Cy5 reporter moiety at its 3′ end. The quencher sequence comprised a 17 nucleotide sequence complementary to the seventeen nucleotide sequence at the 3′ end of the reporter sequence. At its 5′ end, the Quencher sequence carried BHQ2 (“Black Hole Quencher 2”).

As will be appreciated from a consideration of the sequences shown in FIG. 3b, the reporter sequence (comprised of 24 nucleotides) is capable of hybridising to the quencher sequence (comprised of 17 nucleotides) with a seven base overhang at its 5′ end (i.e. the phosphorylated end). Additionally the reporter sequence is capable of hybridising to the WT sequence (comprised of 34 nucleotides) so that the latter has a ten base overhang at its 5′ end.

The above described “hybridisation relationship” between the various sequences is illustrated in FIG. 4 and, as it will be appreciated from the following description, the seven base overhang of the reporter sequence in a duplex formed by hybridisation of the reporter sequence and Quencher sequence (in which the fluorescence of Cy5 is quenched) provides a “target” for the WT, PT1 and PT2 analyte sequences. More specifically, the WT, PT1 and PT2 analyte sequences are such that their 3′ ends will hybridise to the aforementioned seven base overhang of the reporter sequence (in the reporter/Quencher duplex) and that will facilitate full hybridisation of the analyte sequence to the reporter sequence and displacement of the Quencher sequence from the reporter sequence to provide for detectable fluorescent emission from Cy5.

The method of this Example utilises a duplex formed by hybridisation of the reporter and Quencher sequences to “interrogate” samples containing WT, PT1 or PT2 in the presence of λ-exonuclease and provide an amplified signal according to the procedure described more fully above in relation to FIG. 1.

### Experimental Procedure

All oligonucleotides were stored in TE buffer (10 mM Tris-HCl, pH8, 1 mM EDTA) at 10 pmol per μl.

To produce the interrogating duplex, the reporter and quencher oligonucleotides (in TE buffer as above) were mixed together with additional TE buffer in the following ratio reporter/quencher/TE: 4/10/6 μl i.e. 4 pmol of reporter for every 10 pmol of quencher. The mix was then denatured at 95° C. for 5 mins and allowed to hybridise at RT for at least 1 hr. The duplex mixture was then used as an interrogating duplex in a displacement solution experiment.

2 μl of the duplex mixture were added to wells of a black polycarbonate strip. To certain (individual) wells were added 100 fmol of the WT, PT1 and PT2 analyte oligonucleotides (1 μl of their solutions in TE buffer). No analyte oligonucleotides were added to other wells. To each well was added 80 μl of 80% PBS-Tween 0.2% pH9.4-KOH with 5 U of λ-exonuclease and 10 μl of λ-exonuclease buffer (67 mM Glycine-KOH, pH 9.4, 2.5 m M MgCl₂, 50 μg/ml BSA). Liquid volumes in the wells were made up to 100 μl with water.

The reactions were allowed to proceed at room temperature with measurements made after 20 mins and 40 mins.

Cy5 fluorescence was measured on a plate reader against a reporter titration curve of 0, 10, 100 and 1000 fmol of reporter oligonucleotide in the presence of 1×λ-exonuclease buffer per 100 μl of 80% PBS-Tween 0.2% pH9.4-KOH. The readout RFU values were converted to displaced fmol by the use of the titration curve and the 0 mol background was subtracted from all analyte treatments to produce the displacement readout.

### Results

The results of the Example are shown in FIGS. 5(a) and 5(b).

FIG. 5a shows the displacement of the reporter oligonucleotide (minus negative control) for each of the WT, PT1 and PT2 analytes at periods of 20 mins (left-hand bar) and 40 mins (right hand bar). It will be seen that, after 20 mins, the WT analyte had displaced about 74 fmol of reporter oligonucleotide and this figure reached about 146 fmol after 40 minutes. In contrast, the corresponding figures for PT1 were about 35 and about 60 respectively, and those for PT2 were about 18 and 15 respectively.

Therefore FIG. 5(a) shows that displacement amplification as achieved over time, mostly for the WT analyte.

FIG. 5(b) shows the displacement ratios of WT analyte to PT1 and PT2 analytes after both 20 minutes and 40 minutes. For the results after 20 minutes, the left-hand bar is the WT/PT1 ratio and the right-hand bar is the WT/PT2 ratio. Similarly, for the results after 40 minutes.

It will be seen from FIG. 5(b) after 20 minutes that the ratio of WT/PT1 as about 2 and that for WT/PT2 about 4. The corresponding figures for 40 minutes were about 2 and 10 respectively. Generally speaking, therefore, the ratios of WT/PT1 and WT/PT2 increase over time, revealing that amplification is slower for the PT1 and PT2 analytes (than for the WT analyte). This also related to the number of mutations.

### EXAMPLE 2

This Example again demonstrates the specificity of the method of the invention as applied to the WT and PT1 analytes as employed in Example 1.

### Experimental Procedure

All oligonucleotides were stored in TE buffer (10 mM Tris-HCl, pH8, 1 mM EDTA) at 10 pmol per μ1.

To produce the interrogating duplex, the reporter and quencher oligonucleotides (in TE buffer as above) were mixed together with additional TE buffer in the following ratio reporter/quencher/TE: 4/10/6 μl i.e. 4 pmol of reporter for every 10 pmol of quencher. The mix was then denatured at 95° C. for 5 mins and allowed to hybridise at RT for at least 1 hr. The duplex mixture was then used as an interrogating duplex in a displacement solution experiment.

2 μl of the duplex mixture were added to wells of a black polycarbonate strip. To certain (individual) wells were added 10, 10, 50, 100 and 200 fmol of the WT and PT1 analyte oligonucleotides (added in their solutions in TE buffer). No analyte oligonucleotides were added to other wells. To each well was added 80 μl of 80% PBS-Tween 0.2% pH9.4-KOH with 5 U of λ-exonuclease and 10 μl of λ-exonuclease buffer (67 mM Glycine-KOH, pH 9.4, 2.5 m M MgCl₂, 50 μg/ml BSA). Liquid volumes in the wells were made up to 100 μl with water.

The reactions were allowed to proceed at room temperature with measurements made after 20 minutes and 40 minutes.

The procedure was also repeated using the indicated concentrations of the analyte oligonucleotide WT and PT1 but omitting the λ-exonuclease.

Cy5 fluorescence was measured on a plate reader against a reporter titration curve of 0, 10, 100 and 1000 fmol of reporter oligonucleotide per 100 μl of 80% PBS-Tween 0.2% pH9.4-KOH in the presence of 1×λ-exonuclease buffer. The readout RFU values were converted to displaced fmol by the use of the titration curve and the 0 mol background was subtracted from all analyte treatments to produce the displacement readout.

### Results

The results of this Example are shown in FIG. 6.

FIG. 6 shows the amount of reporter sequence (in fmol) displaced by the WT and PT1 analytes at each concentration used. For any one concentration, the result for the WT analyte is the left-hand bar. Considering firstly the results obtained without inclusion of λ-exonuclease, it will be noted that detectable fluorescence was obtained with both the WT and PT1 analyte sequences, although, for any one concentration of analyte sequence, the strongest signal was always obtained with the WT sequence. Similar specificity was retained in the samples incorporating λ-exonuclease.

With the exception of the 10 fmol concentration of PT1 analyte (discussed below), a comparison of the results with and without λ-exonuclease for any particular concentration of analyte sequence shows that amplification of the fluorescence signal had been obtained. Consider, for example, the results for 100 fmol. Without λ-exonuclease, the WT and PT1 sequences displaced ca 93.94 fmol and 36.08 fmol of reporter strand respectively whereas in the presence of the λ-exonuclease the corresponding figures were ca 296.82 and 80.49 respectively. Thus, the WT analyte provided a degree of amplification such that, on average, each WT strand displaced three reporter strands. More generally, the results demonstrate amplification to varying degrees for all concentrations of the WT strand. There was also amplification in the case of the PT1 strand. However, for each concentration tested, the degree of amplification was less for the PT1 strand than the WT strand.

It will be appreciated that a similar experiment using a reporter strand fully complementary to PT1 would provide a set of result in which, for any particular concentration, amplification will be shown to be greater in the case of the PT1 strand than the WT strand.

It will thus be seen that the method of the invention can readily distinguish between a wild-type sequence and one with a point mutation relative thereto.

It will be noted that the amount of reporter sequence displaced (measured in fmol) for the 10 fmol concentration of PT1 analyte in the presence of 5 U of λ-exonuclease was negative. This is attributed to the fact that the interrogating overhang in the interrogating duplex structure was 7 bases long. As such, the background digestion of the interrogating duplex structure was relatively high compared to the displacement achieved by the PT1 analyte. Subtraction of the background displacement from the result obtained for the PT1 run provided the negative result, (probably due to more background in the PT1 run than in the control).#

### EXAMPLE 3

This Example demonstrates use of an interrogating duplex nucleic acid structure with a 16 base overhang in an isothermal reaction carried out at 40° C. to improve sensitivity of the method of the invention.

This Example utilises analyte (WT), reporter and quencher sequences based on the sequence of Neisseria Gonorrhoeae CDS 8 shown in FIG. 7(a).

The analyte, reporter and quencher sequences are shown in FIG. 7(b). The analyte sequence comprised the 45 nucleotides emphasised in the sequence of FIG. 8(a). The reporter sequence (which was phosphorylated at its 5′-end and provided with a Cy5 moiety at its 3′-end) comprised a 35 nucleotide sequence which reading from its 5′ end was complementary to a 35 nucleotide sequence reading from the 3′-end of the analyte. The quencher sequence comprised a 19 nucleotide sequence which (reading from its 5′-end) was complementary with the 19 nucleotides at the 3′-end of the reporter sequence. At its 5′-end, the quencher sequence was provided with a BHQ2 quencher moiety. An interrogating duplex structure prepared by hybridisation of the quencher and reporter sequences had a 16 base overhang at the 5′-end of the reporter.

### Experimental Procedure

All oligonucleotides were stored in TE buffer (10 mM Tris-HCl, pH8, 1 mM EDTA) at 10 pmol per μl.

To produce the interrogating duplex, the reporter and quencher oligonucleotides were mixed in the following ratio reporter/quencher ⅓ i.e. 10 μl of reporter and 30 μl of quencher containing in total 100 and 300 pmol of reporter and quencher respectively. The mix was briefly vortexed and boiled at 95° C. degrees in the dark for 3 mins. 460 μl of 1×PBS, pH7.4, 0.2% Tween was added to the 40 μl oligonucleotide mix, and it was vortexed briefly and incubated at RT in the dark for 1 hr. 50 μl of the mix (containing 10 pmol of reporter oligonucleotide in duplex structure) was added per well of device or plate and another 10 μl of 1×PBS, pH7.4, 0.2% Tween was added per well (to give a 60% PBS dilution in 100 μl of reaction volume) together with 0, 10 amol, 100 amol or 1 fmol of analyte sequence, 1×λ-exonuclease buffer and 10 U of λ-exonuclease in a total of volume of 100 μl volume in made up with H₂O 0.2% Tween at 40° C.

Cy5 fluorescence was measured on a plate reader against a reporter titration curve of 0, 10, 100 and 1000 fmol per 100 μl of 60% PBS-Tween 0.2% pH7.4-KOH, containing 1×λ-exonuclease buffer. The readout RFU values were converted to displaced fmol by the use of the titration curve and the 0 mol background was subtracted from all analyte treatments to produce the displacement readout.

### Results

The results are shown in FIG. 7(c).

As shown in FIG. 7(c) 10 amol of analyte sequence displaced 4.35 fmol, 100 amol displaced 7.78 fmol and 1 fmol displaced 28.61 fmol achieving amplification ratios (reporter molecules displaced/analyte molecules) of 435, 77.8 and 28.61 respectively (FIG. 3.c). It is a common occurrence that amplification ratios drop as analyte concentration increases. This probably due to the fact that the enzyme saturates at higher concentrations of analyte/reporter displaced duplex. The Example does however demonstrate the sensitivity that can be achieved by the method of the invention using (in this case) a 16 base overhang in the interrogating duplex structure, it being noted that a detectable signal was obtained from a sample containing 10 amol (i.e. 10⁻¹⁸ mol) of the analyte sequence.

### EXAMPLE 4

This Example demonstrates the effect on the method of the invention using buffers of different composition.

For the purposes of this Example, a different set of oligonucleotides (“oligos”) was utilised. The sequences of these oligos are shown in Table 2 below.

TABLE 2
Oligo sequences

| Oligo | | Sequence (5′ to 3′) |
|---|---|---|
| name | Function | including modifications |
| RG | Wild type | PO4-CTGTAGAAATCGAAAAATGGGGG |
| | reporter | CTGTGGCTAAAA-Cy5 |
| | | SEQ ID NO: 11 |
| QG | Wild type | BHQ2-TT TTA GCC ACA GCC CCC |
| | quencher | AT SEQ ID NO: 12 |
| RGM* | Mutant | PO4-CTGTAGAAATCGAAAAATCGGGG |
| | reporter | CTGTGGCTAAAA-Cy5 |
| | | SEQ ID NO: 13 |
| QGM* | Mutant | BHQ2-TT TTA GCC ACA GCC CCG |
| | quencher | AT SEQ ID NO: 14 |
| AG | Analyte | TTTTAGCCACAGCCCCCATTTTTCGAT |
| | | TTCTACAG SEQ ID NO: 15 |

*The point mutation is underlined

As shown in Table 2, the oligos included wild type reporter and quencher (RG and QG), the same oligos with a single mutation incorporated (RGM and QGM), and an analyte oligo that is 100% complementary to RG (AG). RG/QG and RGM/QGM were used to create duplexes in order to detect the presence of a particular sequence (the AG sequence in this case) in its wild type form and to discriminate to the same sequence harbouring a single point mutation. Both duplexes created a 16 bases overhang (5′ to 3′ on the reporter) after the reporter and the quencher oligos were hybridised and for the double stranded part of the duplex both oligos had 100% complementarity. Since the mutation is incorporated within the double stranded part of the duplex and in order to maintain 100% complementarity the homologous mutations are incorporated on both RGM and QGM oligos. Sequence alignment for both duplexes are shown in Table 3 below.

TABLE 3
Duplex sequence alignment

RG/QG duplex:
SEQ ID NO: 11
5′-PO4-CTG TAG AAA TCG AAA AAT GGG GGC TGT
GGC TAA AA-Cy5-3′ (RG)
SEQ ID NO: 16
3′-TA CCC CCG ACA CCG ATT TT-BHQ2-5′ (QG)
RGM/QGM duplex (mutation underlined):
SEQ ID NO: 13
5′-PO4-CTG TAG AAA TCG AAA AAT CGG GGC TGT
GGC TAA AA-Cy5-3′ (RGM)
SEQ ID NO: 17
3′-TA GCC CCG ACA CCG ATT TT-BHQ2-5′ (QGM)

PBS-0.2% Tween contains 137 mM NaCl, and it is known from the literature that 25, 50 and 100 mM of sodium chloride inhibits lambda exonuclease activity by 52, 82 and 99% respectively (Little et al 1967). The aim of this Example was (a) to test the reaction in buffers containing various concentrations of sodium chloride, and (b) in buffers which were not formulated with sodium chloride per se but which did contain various concentrations of sodium ions and chloride ions.

Solutions with different concentrations of NaCl (100, 75, 50, 25 and 0% of original PBS NaCl concentration; all buffers contained 0.2% Tween) were prepared as described in Table 4 below. KH₂PO₄ was not added, since its absence does not affect the reaction (data not shown).

TABLE 4

Reaction buffers (mM)

| Chemical | PBS | 100% | 75% | 50% | 25% | 0% |
|---|---|---|---|---|---|---|
| Na2HPO4 | 10 | 10 | 10 | 10 | 10 | 10 |
| KH2PO4 | 1.8 | — | — | — | — | — |
| KCl | 2.7 | 2.7 | 2.7 | 2.7 | 2.7 | 2.7 |
| NaCl | 137 | 137 | 103 | 68.5 | 34 | — |

For the reaction, the AG analyte was used against the RG/QG duplex. The duplex was prepared by mixing together in a single tube 10 pmol of RG and 12 pmol of the QG oligos (in 1 and 1.2 μl of TE respectively), incubating at 95° C. for 3 min, then making the volume up to 50 μl using different buffers. The solution was then left to hybridise for 30 min at room temperature in the dark.

After hybridisation, the duplex solution was pipetted to the wells of a microplate. 10 μl of the equivalent buffer containing 0 or 100 fmol of analyte oligo AG was added to the duplex solution and initial displacement (Pre) was measured on a plate reader. The volume was made up to 100 μl by adding 1 μl (5 U) of lambda exonuclease, 10 μl of 10×lambda exonuclease buffer (1× final concentrations: 67 mM Glycine-KOH, 2.5 mM MgCl2 and 50 μg/ml of BSA, pH 9.4) and 39 μl of the equivalent buffer. Fluorescence was then read immediately (0 min) and at every 10 min after that, up to 30 min. During the assay the samples were incubated at 37° C. in the dark.

Each condition was performed in duplicate and the average of each duplicate for 0 mol of AG was subtracted from the average of each duplicate for 100 fmol of AG for each buffer condition at each time point, providing the net gain in signal due to the presence of analyte. The results are shown in Table 5 below.

TABLE 5

| | Net fluorescence in RFU (100 fmol minus 0 fmol of AG) | | | | |
|---|---|---|---|---|---|
| Buffer | Pre | 0 min | 10 min | 20 min | 30 min |
| PBS | 228 | 272 | 275 | 369 | 500 |
| 100% | 230 | 249 | 225 | 230 | 215 |
| 75% | 227 | 226 | 214 | 219 | 221 |
| 50% | 211 | 252 | 235 | 296 | 352 |
| 25% | 217 | 245 | 464 | 983 | 1356 |
| 0% | 132 | 189 | 831 | 1368 | 1687 |

As can be seen from the results in Table 5, the reaction buffer with 0% NaCl provided the greatest net gain in signal from 10 mins onwards. In the subsequent description the 0% buffer is referred to as Phosphate Buffer (PB).

The procedure described above was repeated with different concentrations of PB i.e. 1×, 2×, 3× and 4×, for which the Na₂HpO₄ and KCl concentrations are shown in Table 6 below.

TABLE 6

| | Reaction buffers (mM) | | | |
|---|---|---|---|---|
| Chemical | 1× PB | 2× PB | 3× PB | 4× PB |
| Na2HPO4 | 10 | 20 | 30 | 40 |
| KCl | 2.7 | 5.4 | 8.1 | 10.8 |

All buffers contained 0.2% Tween.

The purpose of these repeats was to test for any possible beneficial effects of the PB salts to the reaction. The results are shown in Table 7 below which shows the net fluorescence in RFU after subtracting the averages of 0 fmol from 100 fmol of AG for each buffer condition/time point.

TABLE 7

| | Net fluorescence in RFU (100 fmol minus 0 fmol of AG) | | | | |
|---|---|---|---|---|---|
| Buffer | Pre | 0 min | 10 min | 20 min | 30 min |
| 1× PB | 273 | 331 | 1602 | 2217 | 2173 |
| 2× PB | 324 | 378 | 1403 | 2387 | 3017 |
| 3× PB | 317 | 356 | 1020 | 1904 | 2457 |
| 4× PB | 294 | 320 | 583 | 1047 | 1380 |

It can be seen from Table 7 that reaction buffer 2×PB provided the greatest net gain from 20 mins onwards. Therefore since 2×PB produced the best net fluorescence effect, it (2×PB) was chosen as reaction buffer for the purposes of Example 5 below.

### EXAMPLE 5

This Example demonstrates application of the method of the invention to PCR amplicons.

The Examples used the same oligos as listed in Table 2 above together with the primers shown in Table 8 below.

TABLE 8
Oligo sequences

| Oligo | | Sequence (5′ to 3′) |
|---|---|---|
| name | Function | including modifications |
| PO4-FW | Phosphorylated | PO4-CTGTAGAAATCGAAAA |
| | forward primer | ATGGGG SEQ ID NO: 18 |
| REV | Reverse primer | TCTTGGTTCTTTATTTCTAC |
| | | TTGGC SEQ ID NO: 19 |

In brief, an amplicon was generated in a PCR reaction in which one of the primers was phosphorylated on the 5′ end (primer PO4-FW), therefore generating an amplicon with one of the strands phosphorylated on the 5′ end. This strand was recognised by the enzyme (λ-exonuclease) and the phosphorylated strand was digested, releasing the non-phosphorylated strand (the analyte strand). The 3′ end of the analyte strand hybridised to the 5′ end of the reporter and induced displacement. The reporter on the displaced duplex was digested by the enzyme, releasing the analyte for downstream displacement. If analyte to reporter hybridization is not 100% specific the reaction would be delayed or blocked, therefore duplexes of appropriate sequences can be utilised in point mutation analysis.

5 μl of DNA extracted from 12 Neisseria gonorrhoea-positive urine pellet clinical samples derived from 10 ml of urine was used as a template in a PCR reaction (50 μl). The PCR ingredient concentrations per 50 μl of reaction volume are shown in Table 9 and the PCR cycling protocol is shown in Table 10.

TABLE 9

| Reagents | | Concentration/volume/units |
|---|---|---|
| 1× Taq buffer | Tris-HCl | 10 mM |
| | KCl | 50 mM |
| | MgCl2 | 1.5 mM |
| | Template DNA | 5 μl |
| | PO4-FW | 10 pmol |
| | REV | 10 pmol |
| | Taq DNA polymerase | 0.625 U |
| dNTPs | (dATP, dCTP, dGTP, | 200 μM of each |
| | dTTP) | |

TABLE 10

| Number of | | | |
|---|---|---|---|
| cycles | Step | Temp (° C.) | Time (sec) |
| 1× | Initial denaturation | 95 | 30 |
| 30× | Denaturation | 95 | 30 |
| | Annealing | 57 | 60 |
| | Extension | 68 | 60 |
| 1× | Final extension | 68 | 300 |
| Hold | Hold | 12 | ∞ |

After the end of the PCR reaction, 10 μl of each amplicon was used as an analyte against duplexes RG/QG and RGM/QGM. The duplexes and the set-up of the experiment was as above (in 2×PB-0.2% Tween), apart from the fact that 10 μl of amplicon was used instead of 10 μl of reaction buffer plus 0 and 100 fmol of oligo AG. All conditions were in duplicate and net fluorescence was calculated by subtracting the RGM/QGM (mutant duplex) signal from the RG/QG (wild type) signal. The results are included in Table 11 below which shows net fluorescence in RFU after subtracting the averages of the RGM/QGM from the RG/QG signal for Neisseria gonorrhoea-positive sample/time point.

TABLE 11

| | Net fluorescence in RFU (RGM/QGM minus the RG/QG signal) | | | | |
|---|---|---|---|---|---|
| Sample | Pre | 0 min | 10 min | 20 min | 30 min |
| 1 | −276 | −151 | 1744 | 3482 | 4891 |
| 2 | −261 | −108 | 1934 | 3948 | 5416 |
| 3 | −232 | −214 | 159 | 601 | 1096 |
| 4 | −243 | −237 | 245 | 704 | 1163 |
| 5 | −257 | 291 | 2344 | 4146 | 5566 |
| 6 | −257 | 19 | 1359 | 2580 | 3440 |
| 7 | −256 | 355 | 2476 | 4184 | 5450 |
| 8 | −255 | −8 | 2217 | 4281 | 5719 |
| 9 | −218 | −116 | 2205 | 4109 | 5652 |
| 10 | −220 | −5 | 1167 | 2207 | 2976 |
| 11 | −233 | −71 | 1088 | 2166 | 2954 |
| 12 | −222 | 25 | 1007 | 1875 | 2637 |

In all cases net fluorescence increased with time, indicating that the analyte sequence in question did not contain the mutation introduced in duplex RGM/QGM. The same effect was also observed by the use of oligo AG (wild type oligo) vs the RG/QG and RGM/QGM duplexes (data not shown). Because the RGM/QGM duplex exhibited a slightly higher background fluorescence than the RG/QG duplex net gain was negative for the Pre and some of the 0 min conditions. However, the net gain turned positive soon after λ-exonuclease was introduced to the reaction (10 mins) and kept increasing with time

### REFERENCES

Little, J. W. Lehman, I. R. Kaiser, A. D. (1967) An exonuclease induced by bacteriophage λ I. preparation of the crystalline enzyme. The Journal of Biological Chemistry. 242(4): 672-678
P G Mitsis and J G Kwaqh (1999) Characterization of the interaction of lambda exonuclease with the ends of DNA. Nucleic Acids Res. 27(15): 3057-3063.
C. S. Karapetis (2008) K-ras Mutations and Benefit from Cetuximab in Advanced Colorectal Cancer. The New England Journal of Medicine, 359 (17):1757-1765.
Rogers, G. S. and Weiss, B. (1980). L. Grossman and K. Moldave (Ed.), Methods Enzymol. 65, 201-211. New York: Academic Press.

1. A method of analysing for a single strand target nucleic acid sequence in a target nucleic acid present, or potentially present, in a sample the method comprising the steps of:
(i) providing an interrogating duplex nucleic acid structure which comprises
(a) a first reporter strand which is specifically hybridizable to the single strand target nucleic acid sequence in the target strand (if present in the sample) and which is tagged at or towards one end thereof with a reporter moiety capable of providing a detectable signal, said reporter strand being configured such that in a reporter/target duplex structure formed by hybridisation of the first reporter strand and the single strand target nucleic acid sequence the reporter strand may be selectively enzymatically digested from its end opposite the reporter moiety to release the hybridised single strand target nucleic acid sequence and the reporter moiety; and
(b) a second, displaceable strand shorter than said first reporter strand and hybridised thereto to form the interrogating duplex nucleic acid structure in which the reporter strand provides an interrogating overhang,
(ii) providing an enzyme capable of effecting said selective enzymatic digestion of the reporter strand in a duplex structure comprised of the reporter strand and target nucleic acid sequence, and
(iii) effecting the analysis under conditions such that the single strand target nucleic acid sequence, if present, displaces the second strand from the interrogating duplex nucleic acid structure and hybridises to the reporter strand with subsequent enzymatic digestion of the reporter strand, and
(iv) directly or indirectly detecting for the presence of reporter moiety.

2. A method as claimed in claim 1 wherein the single strand target nucleic acid is present in the sample.

3. A method as claimed in claim 1 effected isothermally.

4. A method as claimed in claim 1 wherein the reporter/target duplex structure formed by hybridisation of the first reporter strand and the single strand target nucleic acid has a blunt end and enzymatic digestion of the reporter strand proceeds from that end of the duplex structure.

5. A method as claimed in claim 4 wherein the target nucleic acid strand is longer than the reporter strand and forms a “tail” in the reporter/target duplex structure.

6. A method as claimed in claim 5 wherein the tail has a maximum length of 200 bases.

7. A method as claimed in claim 4 wherein the blunt end is at the 5′-end of the reporter strand.

8. A method as claimed in claim 1 wherein the 5′-end of the reporter strand is phosphorylated.

9. A method as claimed in claim 8 wherein the enzyme is A-exonuclease.

10. A method as claimed in claim 9 wherein the target nucleic acid is produced in a PCR reaction effected using a primer with a 5-phosphorylated end.

11. A method as claimed in claim 1 wherein the overhang in the interrogating duplex nucleic acid structure has 5 to 20 bases.

12. A method as claimed in claim 11 wherein the overhang in the interrogating duplex nucleic acid structure has 7 to 20 bases.

13. A method as claimed in claim 12 wherein the overhang in the interrogating duplex nucleic acid structure has 15 to 17, preferably 16, bases.

14. A method as claimed in claim 1 wherein the enzyme is A-exonuclease, the reporter strand has a 5′-phosphorylated end, the interrogating overhang has 15 to 20 bases, and the reaction is effected isothermally.

15. A method as claimed in claim 14 wherein, in the reporter/target duplex structure the target nucleic acid strand has a tail having a maximum length of 100 bases.

16. A method as claimed in claim 14 wherein the reaction is effected at a temperature of 35° C. to 40° C., preferably 37° C.

17. A method as claimed in claim 14 wherein the interrogating overhang has a length of 15 to 17 bases.

18. A method as claimed in claim 1 wherein the reporter moiety is a luminescent moiety.

19. A method as claimed in claim 18 wherein the second displaceable strand is provided with a quencher moiety to quench luminescence of the reporter moiety in the interrogating duplex nucleic acid structure.

20. A method as claimed in claim 1 wherein the reporter moiety is an enzyme capable of detecting a signal upon reaction with a substrate.

21. A method as claimed in claim 1 effected in the liquid phase.

22. A method as claimed in claim 1 wherein the interrogating duplex nucleic acid structure is immobilised on a solid phase by virtue of the second displaceable strand being attached to the solid phase.

23. A method as claimed in claim 22 effected in an assay device having a reaction region at which the immobilised interrogating duplex nucleic acid structure is provided and a downstream detection region at which signal resulting from the reporter moiety is detected, said liquid sample flowing from the reaction region to the detection region during the method.

24. A method as claimed in claim 23 wherein the assay device has an upstream sampling region to which the liquid sample is applied, the liquid sample flowing from the sampling region and through the reaction to the detection region during the method.

25. A method as claimed in claim 1 wherein the sample is a liquid sample.

26. A method as claimed in claim 1 effected in an aqueous phosphate-based buffer having a concentration of less than 80 mM of each of sodium and chloride ions.

27. A method as claimed in claim 26 wherein the buffer has a concentration of 20 to 50 mM sodium ions and 2 to 7 mM chloride ions.

28. (canceled)

29. A method as claimed in claim 1 wherein the target nucleic acid sequence is AATTGGTCGCATAACAATAGAAATATATGCCAAG (SEQ ID NO: 20) or a variant thereof having one or two point mutations.

30. A procedure for identifying a target nucleic acid sequence in a target nucleic acid strand in a sample, said target nucleic acid sequence either having a first sequence or a second sequence differing from the first sequence by a base change, wherein the procedure comprises the steps of:
(i) effecting method of claim 1 with a reporter strand including a sequence which is fully complementary to said first sequence; and
(ii) effecting the method with a reporter strand including a sequence which is fully complementary to said second sequence; and
(iii) comparing the results of steps (i) and (ii) to determine whether the target sequence is said first sequence or said second sequence.
